(19) United States
(12) Patent Application Publication
(10) Pub. No.: US 2010/0249116 A1
(43) Pub. Date: Sep. 30, 2010

(54) Compositions and Methods for Treating Intoxications

(76) Inventors: Nikolay Vasilievich Goncharov, Leningradsky Region (RU); Anatoliy Vasilievich Kuznetsov, Leningradsky Region (RU); Lidia Mikhailovna Glashkina, Leningradsky Region (RU); Andrey Stanislavovich Radilov, Leningradsky Region (RU)

Correspondence Address: STEPTOE & JOHNSON LLP, 1330 CONNECTICUT AVENUE, N.W., WASHINGTON, DC, 20036, US

(21) Appl. No.: 12/532,761

(22) PCT Filed: Mar. 23, 2007
(86) PCT No.: PCT/RU0207/000142

§ 371 Date: May 21, 2010

(51) Int. Cl. A61K 31/5415; A61P 39/00

(52) U.S. Cl. 514/224.8

(57) ABSTRACT

Compositions and methods for treating intoxication by poisonous agents are described. In one aspect the pharmaceutical compositions include a phenothiazine compound, a dioic acid compound and a pharmaceutically acceptable carrier. In another aspect the pharmaceutical composition can include a phenothiazine compound, a nitroester compound, ethanol and a pharmaceutically acceptable carrier.

### TECHNICAL FIELD

This invention relates generally to compositions and methods for treating intoxication by poisonous agents.

### BACKGROUND

The pathogenic action of intoxication by fluoroacetates, ethylene glycol, cyanide and other poisonous agents is often the development of hypoxic or ischemic states in an intoxicated human or animal.

Fluoroacetates and other monofluorides are highly toxic compounds. Their actions characteristically involves a latent period, which for humans is from half an hour to several hours even with exposure to lethal doses (Goncharov et al., J. Appl. Toxicol. 26:148-161 (2006), which is incorporated by reference in its entirety). The sodium salt of fluoroacetate has been used in some countries for pest control. Accidental or intentional acute intoxications can occur during exposure to a stock solution during formulation, dermal or respiratory exposure during application of baits. Further, some fluorinated compounds such as anticancer agents, narcotic analgesics, pesticides or industrial chemicals, metabolize to fluoroacetate as an intermediate product. Fluoroacetate undergoes a series of metabolic conversions that result in the synthesis of fluorocitrate which acts by blocking energy production through inhibition of the Krebs cycle. Antidotal therapy for fluoroacetate intoxication thus far has been aimed at preventing fluorocitrate synthesis and aconitase blockade in mitochondria, and at providing citrate outflow from this organelle. The most acceptable antidote for the past six decades has been ethanol.

Cyanide (CN⁻), a fast acting toxic compound, is frequently used in suicides, homicide, and chemical warfare (see, for example, Salkowski et al., Vet. Hum. Toxicol. 36:455-466 (1994) and Borowitz et al., B. Somani (Ed.), Chemical Warfare Agents, Academic Press, New York, pp. 209-236 (1992), which is incorporated by reference in its entirety). Cyanide toxicity can arise from a variety of sources, e.g., from inhalation of smoke produced by the pyrolysis of plastics or nitrile-based polymer fibers, materials that are commonly used in construction and for furniture manufacture. Cyanide toxicity can also occur from ingestion of plant extracts containing cyanogenic glycosides, or from inhalation of airborne vapors encountered in industrial or occupational settings. The systemic toxic effect of cyanide has been attributed mainly to its binding to the ferric iron in cytochrome c oxidase. The reaction forms a stable but reversible complex and subsequently disrupts cellular energy production. The reduction of cellular oxygen consumption results in an increase in venous oxygen partial pressure. The classic antidotal action for cyanide poisoning involves inhalation of amyl nitrite, followed by intravenous injection of sodium nitrite and sodium thiosulfate (Chen et al., Proc. Soc. Exp. Biol. Med. 31:250-252 (1933), which is incorporated by reference in its entirety). This procedure is still used clinically worldwide, including the United States (see, for example, Dreisbach, in Handbook of poisoning: Diagnosis and treatment, 12th edition, Lange Med. Publications., Los Altos, Calif., p. 251 (1987), which is incorporated by reference in its entirety).

Glycol poisoning, particularly ethylene glycol (EG) poisoning, is a serious, often fatal condition. Glycol poisoning is common among children and animals in particular, due to its presence in high concentrations in automobile antifreeze and other common household products. An unknowing child or animal can accidentally drink the glycol containing liquid usually because of its sweet odor and taste, and unless treatment is begun promptly after ingestion, permanent damage or death will result depending on the amount ingested. Following ingestion, ethylene glycol is first hepatically metabolized to glycoaldehyde by alcohol dehydrogenase. Glycoaldehyde is then oxidized to glycolic acid, glyoxylic acid and finally oxalic acid. Thus, the acute toxicity of ethylene glycol can proceed through three stages, each associated with a different metabolite: central nervous system depression (ethylene glycol), cardiopulmonary effects associated with metabolic acidosis (glycolic acid), and ultimately renal toxicity (oxalic acid). Toxicity of ethylene glycol can depend on the total amount consumed and the effectiveness of therapeutic interventions. Current antidotes/treatments for glycol poisoning are limited. Ethylene glycol metabolism can be inhibited by the use of antidotes that inhibit alcohol dehydrogenase. Historically, this has been done with intoxicating doses of ethanol. At a sufficiently high concentration, ethanol saturates alcohol dehydrogenase, preventing it from acting on ethylene glycol, thus allowing the latter to be excreted unchanged by the kidneys. However, ethanol therapy is complicated by its own inherent toxicity, and the need to carefully monitor serum ethanol concentrations and adjust the rate of administration. A recent alternative to ethanol therapy is fomepizole, or 4-methylpyrazole. Like ethanol, fomepizole inhibits alcohol dehydrogenase; however it does so without producing serious adverse effects (Brent J. Drugs. 2001; 61(7):979-88; Corley R A, McMartin K E. Toxicol Sci. 2005; 85(1):491-501, the entire contents of which is hereby incorporated by reference).

### SUMMARY

In general, pharmaceutical compositions and methods for treating subjects suffering from intoxication by poisonous agents such as fluoroacetate, cyanide, ammonia and ethylene glycol are described.

In one aspect, a pharmaceutical composition includes a phenothiazine compound, a dioic acid compound and a pharmaceutically acceptable carrier. The phenothiazine compound can be methylene blue. The dioic acid compound can be a glutamate salt. The glutamate salt can be sodium glutamate.

In another aspect, a pharmaceutical composition can include a phenothiazine compound, a nitroester compound, ethanol and a pharmaceutically acceptable carrier. The phenothiazine compound can be methylene blue. The nitroester compound can be glycerol trinitrate. The pharmaceutical composition can further include a pyrazole. The pyrazole can be 4-methylpyrazole. The pharmaceutical composition can further include a dioic acid compound. The dioic acid compound can be a glutamate salt. The glutamate salt can be sodium glutamate. The dioic acid compound can be a 2-oxoglutrate salt. The 2-oxoglutrate salt can be sodium 2-oxoglutrate. The dioic acid compound can be an aspartate salt. The dioic acid compound can be a succinate salt. The pharmaceutical composition can further include a monocarboxylic acid compound. The monocarboxylic acid compound can be a pyruvate salt. The pyruvate salt can be sodium pyruvate.

In a further aspect, a method of treating fluoroacetate intoxication can include administering to a subject in need thereof an effective amount of a composition that includes a phenothiazine compound, a nitroester compound and ethanol. The phenothiazine compound can be methylene blue. The nitroester compound can be glycerol trinitrate. The composition can further include a dioic acid compound. The dioic acid compound can be a glutamate salt. The glutamate salt can be sodium glutamate. The dioic acid compound can be a 2-oxoglutarate salt. The 2-oxoglutarate salt can be sodium 2-oxoglutarate. The dioic acid compound can be an aspartate salt. The dioic acid compound can be a succinate salt. The composition can further include a monocarboxylate salt. The monocarboxylate salt can be a pyruvate salt. The pyruvate salt can be sodium pyruvate. The phenothiazine compound can be administered subcutaneously. The phenothiazine compound can be administered 45-75 minutes and 100-140 minutes after intoxication. The phenothiazine compound can be administered 5-15 minutes and 100-140 minutes after intoxication. The nitroester compound and ethanol can be administered intraperitoneally. The nitroester compound and ethanol can be administered 5-15 minutes, 50-70 minutes and 100-140 minutes after intoxication. The phenothiazine compound, the nitroester compound and ethanol can further be administered 20-28 hours and 40-56 hours after intoxication.

In another aspect, a method of treating ethylene glycol intoxication can include administering to a subject in need thereof an effective amount of a composition including a phenothiazine compound, a nitroester compound and ethanol. The phenothiazine compound can be methylene blue. The nitroester compound can be glycerol trinitrate. The phenothiazine compound can be administered subcutaneously. The phenothiazine compound can be administered 45-75 minutes and 100-140 minutes after intoxication. The phenothiazine compound can be administered 5-15 minutes and 100-140 minutes after intoxication. The nitroester compound and ethanol can be administered intraperitoneally. The nitroester compound and ethanol can be administered 5-15, 50-70 and 100-140 minutes after intoxication. The phenothiazine compound, the nitroester compound and ethanol can further be administered 20-28 hours and 40-56 hours after intoxication. The composition can further include a pyrazole. The pyrazole can be 4-methylpyrazole. The pyrazole can be administered subcutaneously. The pyrazole can be administered 5-15 minutes and 100-140 minutes after intoxication. The phenothiazine compound, the nitroester compound and ethanol can be administered 45-75 minutes and 20-28 hours after intoxication.

In a further aspect, a method of treating cyanide intoxication can include administering to a subject in need thereof an effective amount of a composition including a phenothiazine compound, a nitroester compound and ethanol. The phenothiazine compound can be methylene blue. The nitroester compound can be glycerol trinitrate. The phenothiazine compound, the nitroester compound and ethanol can be administered intraperitoneally. The phenothiazine compound, the nitroester compound and ethanol can be administered 0.5-2 minutes and 25-45 minutes after intoxication.

In another aspect, a method of treating ammonia intoxication can include administering to a subject in need thereof an effective amount of a composition including a phenothiazine compound and a dioic acid compound. The phenothiazine compound can be methylene blue. The dioic acid compound can be a glutamate salt. The glutamate salt can be sodium glutamate. The phenothiazine compound can be administered subcutaneously. The dioic acid compound can be administered intraperitoneally.

Other aspects, features, and objects will be apparent from the description and the claims.

### DETAILED DESCRIPTION

Advantageously, the repeated introduction of a specific combination of compounds can have therapeutic effect on subjects suffering from intoxication by fluoroacetates, ethylene glycol, cyanide or other poisonous agents that result in hypoxic or ischemic states.

A composition can include specific combinations of compounds such as a phenothiazine compound, a dioic acid compound and/or a monocarboxylic (or monocarboxylated) compound, a nitroester compound, and/or an alkyl alcohol.

A phenothiazine compound can be a substituted or unsubstituted phenothiazine compound, or salt thereof. The phenothiazine can be substituted with one or more substituents independently selected from halogen, hydroxyl, alkoxy, amino, alkylamino, dialkylamino, or alkyl. Examples of phenothiazine compounds can include but are not limited to chlorpromazine, fluphenazine, mesoridazine, perphenazine, prochlorperazine, promazine, thioridazine, trifluoperazine, triflupromazine or methylene blue. The methylene blue can be methylene blue or a derivative such as azure A, B, C and thionine.

A dioic (or di-carboxylic) acid compound can be a C₂-C₆ dioic acid, or a salt thereof. For example, the dioic acid compound can have the formula HOOC—(CR¹R²)ₙ—COOH, in which n is 0, 1, 2, 3, 4, 5 or 6, and each of R¹ and R² is, independently, H, halogen, C₁-C₄ alkyl, amino, substituted amino, thio, or hydroxyl, or a bond (with an adjacent R¹ or R² to form a double or triple bond), or R¹ and R² together are oxo, C₁-C₄ alkylidene or imine. Examples of a dioic acid compound include glutamic acid, aspartic acid, 2-oxoglutaric acid, malonic acid, succinic acid, adipic acid, pilemic acid, suberic acid. The salt can be a mono-sodium salt, a mono-lithium salt, a mono-potassium, a di-sodium salt, a di-lithium salt, a di-potassium salt, a calcium salt, a magnesium salt or other pharmaceutically acceptable salt. For example, the dioic compound can be sodium glutamate, sodium 2-oxoglutarate, or sodium aspartate.

A monocarboxylic (or monocarboxylated) compound can be a C₂-C₆ monocarboxylic acid, or a salt of thereof. For example, the monocarboxylic acid compound can have the formula C(R³)₃—(CR¹R²)ₙ—COOH, in which n is 0, 1, 2, 3, 4, 5 or 6, and each of R¹, R² and R³ is, independently, H, halogen, C₁-C₄ alkyl, amino, substituted amino, thio, or hydroxyl, or a bond (with an adjacent R¹ or R² to form a double or triple bond), or R¹ and R² together are oxo, C₁-C₄ alkylidene or imine. Examples of a monocarboxylic acid compound include pyruvic acid, acetic acid, lactic acid. The salt can be a mono-sodium salt, a mono-lithium salt, a mono-potassium, a di-sodium salt, a di-lithium salt, a di-potassium salt, a calcium salt, a magnesium salt or other pharmaceutically acceptable salt. For example, the monocarboxylic compound can be sodium pyruvate, sodium acetate, or sodium lactate.

A nitroester compound can be a monoalcohol or polyol, for example, dialcohol, trialcohol, tetraalcohol, pentaalcohol or hexaalcohol, having one or more nitroester groups. For example, the nitroester compound can be pentaerythritol tetranitrate, glycerol trinitrate or isosorbide dinitrate.

The alkyl alcohol can be ethanol.

In one embodiment, a composition can include a phenothiazine compound and a dioic acid compound. For example, the composition includes methylene blue and sodium glutamate.

In another embodiment, a composition can include a phenothiazine compound, ethyl alcohol and a nitroester compound. For example, the composition includes methylene blue, ethanol and glycerol trinitrate.

In a further embodiment, a composition can include a phenothiazine compound, ethyl alcohol, a nitroester compound, and a dioic acid compound or monocarboxylic compound, or combinations thereof. For example, the composition includes methylene blue, ethanol, glycerol trinitrate, and sodium glutamate or 2-oxoglutarate, pyruvate, succinate, aspartate.

The compounds in each unique composition can be used in the form of pharmaceutically acceptable salts derived from inorganic or organic acids and bases. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pamoate, pectinate, persulfate, 3-phenyl-propionate, picrate, pivalate, propionate, succinate, tartrate, thiocyanate, tosylate or undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth. Also, the basic nitrogen-containing groups can be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides or others. Water or oil-soluble or dispersible products are thereby obtained.

A pharmaceutical composition can include an effective amount of a combination of compounds described herein. An effective amount is defined as the amount which is required to confer a therapeutic effect on the treated subject, and will depend on a variety of factors, such as the nature of poisonous agent, the size of the subject, the goal of the treatment, the nature of the pathology to be treated, the specific pharmaceutical composition used, and the judgment of the treating physician. For reference, see Freireich et al., Cancer Chemother. Rep. 1966, 50, 219 and Scientific Tables, Geigy Pharmaceuticals, Ardsley, N.Y., 1970, 537. Dosage levels of between about 0.001 and about 100 g/kg body weight per day, preferably between about 0.01 and about 10 g/kg body weight per day of the active ingredient compound are useful. The effective amount can also vary, as recognized by those skilled in the art, depending on the route of administration, excipient usage, and the possibility of co-usage, pre-treatment or post-treatment, with other therapeutic treatments including the use of other antidotes. The effective amount of active ingredient can also depend upon the therapeutic or prophylactic agent, if any, with which the ingredient is co-administered. A subject can include a human or a non-human such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. A subject can also include non-mammals such as birds, and the like.

In one embodiment, a method of treating sodium fluoroacetate intoxication can include administering to a subject in need thereof, an effective amount of a composition that includes a phenothiazine compound and a dioic acid compound. The phenothiazine compound can be methylene blue and the dioic acid compound can be sodium glutamate.

In another embodiment, a method of treating sodium fluoroacetate intoxication can include administering to a subject in need thereof, an effective amount of a composition that includes a phenothiazine compound, a nitroester compound and an alkyl alcohol. The phenothiazine compound can be methylene blue. The nitroester compound can be glycerol trinitrate. The alkyl alcohol can be ethanol. In a further embodiment, a method of treating sodium fluoroacetate intoxication can include administering to a subject in need thereof, an effective amount of a composition that includes a phenothiazine compound, a nitroester compound, an alkyl alcohol and a dioic acid compound. The phenothiazine compound can be methylene blue. The nitroester compound can be glycerol trinitrate. The alkyl alcohol can be ethanol. The dioic acid compound can be sodium glutamate or 2-oxo-glutarate or aspartate or succinate. In another embodiment, a method of treating sodium fluoroacetate intoxication can include administering to a subject in need thereof, an effective amount of a composition that includes a phenothiazine compound, a nitroester compound, an alkyl alcohol and a monocarboxylate compound. The phenothiazine compound can be methylene blue. The nitroester compound can be glycerol trinitrate. The alkyl alcohol can be ethanol. The monocarboxylate compound can be pyruvate or acetate or lactate.

In one embodiment, a method of treating ethylene glycol intoxication can include administering to a subject in need thereof, an effective amount of a composition that includes a phenothiazine compound, a nitroester compound and an alkyl alcohol. The phenothiazine compound can be methylene blue. The nitroester compound can be glycerol trinitrate. The alkyl alcohol can be ethanol.

In another embodiment, a method of treating potassium cyanide intoxication can include administering to a subject in need thereof, an effective amount of a composition that includes a phenothiazine compound, a nitroester compound and an alkyl alcohol. The phenothiazine compound can be methylene blue. The nitroester compound can be glycerol trinitrate. The alkyl alcohol can be ethanol.

In a further embodiment, a method of treating ammonia intoxication can include administering to a subject in need thereof, an effective amount of a composition that includes a phenothiazine compound and a dioic acid compound. The phenothiazine compound can be methylene blue. The dioic acid compound can be sodium glutamate or 2-oxo-glutarate or succinate.

In the course of treatment, the compound in each specific composition can be administered together or sequentially. Each composition can be administered initially, periodically, or repeatedly, to a subject in need thereof, before or immediately after acute intoxication or within 0 minutes, 5 minutes, 10 minutes, 15 minutes, 20 minutes, 25 minutes, 30 minutes, 35 minutes, 40 minutes, 45 minutes, 50 minutes, 55 minutes, 1 hour, 90 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours or 24 hours after intoxication. The compound in each specific composition can be administered 0-0.5 minutes, 0.5-2 minutes, 2-5 minutes, 5-10 minutes, 5-15 minutes, 15-25 minutes, 25-45 minutes, 45-75 minutes, 75-100 minutes, 100-140 minutes, 20-28 hours or 40-56 hours after intoxication. For example, the phenothiazine compound can be administered 60 minutes and 120 minutes after intoxication. The phenothiazine compound can be administered 10 minutes and 120 minutes after intoxication. The nitroester compound and ethanol can be administered 10 minutes, 60 minutes and 120 minutes after intoxication. The phenothiazine compound, the nitroester compound and ethanol can be administered 1 minute and 30 minutes after intoxication. The phenothiazine compound, the nitroester compound and ethanol can be administered 1 hour and 24 hours after intoxication. The phenothiazine compound, the nitroester compound and ethanol can be administered 24 hours and 48 hours after intoxication.

The composition can be administered to a subject in need thereof, less than 96 hours, less than 72 hours, less than 48 hours, less than 36 hours, less than 30 hours, less than 24 hours, less than 18 hours, less than 12 hours, less than 6 hours, less than 3 hours or less than 1 hour after intoxication. The composition can be administered to a subject suffering from chronic intoxication ten times a day, nine times a day, eight times a day, seven times a day, six times a day, five times a day, four times a day, three times a day, twice a day, daily, every two days, every 3 days, every 4 days, every 5 days, every 6 days, weekly, biweekly, monthly or as frequently as needed.

Antidotes that are specific to the type of intoxication can be co-administered with the composition either simultaneously or sequentially. Antidotes can be administered to a subject in need thereof before or after administering the composition. Examples of antidotes can include, but are not limited to, amyl nitrite, sodium nitrite, sodium thiosulfate or vitamin B₁₂ for cyanide poisoning; pyrazole such as 4-methylpyrazole for ethylene glycol intoxication.

The combinations of compounds can be formulated into pharmaceutical compositions that can be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional, intracranial injection or infusion techniques.

The pharmaceutical composition can include different combinations of compounds or pharmaceutically acceptable derivatives thereof, together with any pharmaceutically acceptable carrier. The term “carrier” as used herein includes acceptable adjuvants and vehicles. Pharmaceutically acceptable carriers that can be used include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol or wool fat.

The pharmaceutical composition can be in the form of a sterile injectable preparation, for example a sterile injectable aqueous or oleaginous suspension. This suspension can be formulated according to techniques known in the art using suitable dispersing or wetting agents and suspending agents. The sterile injectable preparation can also be a sterile injectable solution or suspension in a non-toxic parenterally-acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or di-glycerides. Fatty acids, such as oleic acid and its glyceride derivatives are useful in the preparation of injectables, as do natural pharmaceutically-acceptable oils, such as olive oil or castor oil, especially in their polyoxyethylated versions. These oil solutions or suspensions can also contain a long-chain alcohol diluent or dispersant.

The pharmaceutical compositions can be orally administered in any orally acceptable dosage form including, but not limited to, capsules, tablets, aqueous suspensions or solutions.

In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents can also be added.

The pharmaceutical composition can be administered in the form of suppositories for rectal administration. These can be prepared by mixing the agent with a suitable non-irritating excipient which is solid at room temperature but liquid at the rectal temperature and therefore will melt in the rectum to release the drug. Such materials include cocoa butter, beeswax or polyethylene glycols.

The pharmaceutical composition can be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches can also be used.

For topical applications, the pharmaceutical composition can be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax or water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol or water.

For ophthalmic use, the pharmaceutical composition can be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutical compositions can be formulated in an ointment such as petrolatum.

The pharmaceutical composition can also be administered by nasal aerosol or inhalation through the use of a nebulizer, a dry powder inhaler or a metered dose inhaler. Such compositions are prepared according to techniques well-known in the art of pharmaceutical formulation and can be prepared as solutions in saline, employing benzyl alcohol or other suitable preservatives, absorption promoters to enhance bioavailability, fluorocarbons, and/or other conventional solubilizing or dispersing agents.

The following examples are for the purpose of illustration only and are not intended to be limiting.

### EXAMPLES

### Materials and Methods

The compounds and conditions for administration were tested in experimental animals such as rats and mice. The compounds were freshly prepared as aqueous solutes (not more than 1 day before administration). Sodium fluoroacetate (SFA) was synthesized according to Berhmann et al., J. Chem. Soc. (London), p. 3786-3788 (1953) at the Research Institute of Hygiene, Occupational Pathology and Human Ecology (St. Petersburg, Russia). According to gas chromatography-mass spectrometry (GC-MS) and elemental analysis data, the purity of the synthesized sample was no less than 98%. Methylene blue (MB), Sodium Glutamate (SG), and 4-Methyl-Pyrazol (4 MP) were obtained from Sigma, 1% solution of glycerol trinitrate (GT) in ethanol (ET) was obtained from Merck, sodium pyruvate was obtained from Serva. Ethylene Glycol (EG), 2-oxo-glutarate, Ammonia Chloride (AC) and Potassium Cyanide (KCN) of chemical grade purity were purchased from Ekros, Russia.

Outbred and Wistar rats, male and female, and outbred mice were purchased from Rappolovo breeding facility (Leningradsky Region) and kept in the Institute's vivarium. 5-6 animals were housed per cage (temperature of 21 C±2 C, relative humidity of not less than 30%). The animals had free access to standard laboratory rodent pellet diet (Laboratorsnab Company, Moscow Region) and tap water. At least 2 weeks acclimation and quarantine period was allowed before starting experiments. Rats of 2-6 months old and weighing 200-400 g were used in the experiments. To minimize seasonal or age fluctuations, the key experiments were performed with rats of the same sex, purchased from the same source and about the same age. Those experiments were performed within a short period of time (3-4 weeks). There were usually 3-4 rats per group and 3-4 groups in a single experiment, which could be repeated if necessary to obtaining statistically significant data. Control and treated rats were observed for a period of 2 weeks, and cumulative mortality in each group was recorded. Toxicity parameters were calculated by the probit analysis according to the Litchfield-Wilcoxon method, with the help of a special software programme.

SFA dissolved in distilled water was administered perorally (intragastric injection with a special stainless steel probe connected with 1 ml glass syringe) at 0.2 ml per 100 g of rat body weight, after 7-9 hours fasting.

A combination of two compounds: 1) aqueous solution of 5 mg/ml methylene blue (MB), administered subcutaneously at a dose of 5 mg/kg; 2) aqueous solution of 130 mg/ml sodium glutamate (SG), administered intraperitoneally at a dose 250 mg/kg, was administered 10 and 120 min after the acute poisoning with sodium fluoroacetate (SFA).

The combination of three compounds: aqueous solution of MB (dose 5 mg/kg administered subcutaneously) and aqueous solution of 25-50% Ethanol (ET) and 0.25-0.5% glycerol trinitrate (GT) (corresponding doses for pure Ethanol (ET) and glycerol trinitrate (GT) were 1 g/kg and 10 mg/kg administered intraperitoneally); was administered in 10, 60 and 120 min (ET and GT) or 60 and 120 min (MB) after the poisoning with SFA.

The combination developed for the case of potassium cyanide (KCN) intoxication: methylene blue (MB) and Ethanol (ET) with glycerol trinitrate (GT) was mixed together and introduced intraperitoneally at 1 and 30 min after the poisoning. The dose of MB was enhanced up to 10 mg/kg.

The combination of four compounds: aqueous solutions of methylene blue (MB) (dose 5 mg/kg administered subcutaneously) and 25% ET, 0.25% GT, and 12.5-25 mg/ml sodium glutamate (SG), so that corresponding doses for pure Ethanol (ET), glycerol trinitrate (GT), and sodium glutamate (SG) were 1 g/kg, 10 mg/kg, and 50-100 mg/kg, given intraperitoneally; was administered in 10, 60 and 120 min, 24 h and 48 h (for ET, GT, and SG), and 10 and 120 min, 24 h and 48 h (for MB) after the poisoning with SFA.

To test the effectiveness of the preparations under acute intoxication with sodium fluoroacetate (SFA), the combination of methylene blue and sodium glutamate was administered 10 and 120 min after the acute poisoning with SFA and was found to have a coefficient of efficiency for LD50 (K_{LD50}) on the level of 2.5 (Table 1).

TABLE 1
Assessment of the effectiveness of methylene
blue (MB) and sodium glutamate (SG) combination
under acute SFA intoxication of female rats

| | | Number | | Probit analysis |
|---|---|---|---|---|
| Dose, | | of | Death | using Litchfield- |
| mg/kg | Therapy | rats | rate | Wilcoxon method |
| 1.17 | No | 6 | 1 | LD16 = 1.179 |
| 1.4 | No | 6 | 3 | LD50 = 1.368 |
| 1.75 | No | 6 | 6 | (1.215 ÷ 1.540) |
| | | | | LD84 = 1.586 |
| 3.0 | Combination (MB + | 6 | 0 | LD16′ = 3.199 |
| | SG) 10\grave{ } + 2 h | | | LD50′ = 3.476 |
| | after poisoning | | | (3.292 ÷ 3.670) |
| 3.5 | Combination (MB + | 18 | 9 | LD84′ = 3.778 |
| | SG) 10\grave{ } + 2 h | | | |
| | after poisoning | | | |
| 4.0 | Combination (MB + | 6 | 6 | |
| | SG) 10\grave{ } + 2 h | | | |
| | after poisoning | | | |

| Coefficients of efficiency: | K_{LD16} = LD16′/ |
|---|---|
| | LD16 = 2.7 |
| | K_{LD50} = LD50′/ |
| | LD50 = 2.5 |
| | K_{LD84} = LD84′/ |
| | LD84 = 2.3 |

The combination of three compounds: methylene blue (MB) (dose 5 mg/kg administered subcutaneously) and aqueous solution of 25-50% ethanol (ET) and 0.25-0.5% glycerol trinitrate (GT) (corresponding doses for pure ET and GT were 1 g/kg and 10 mg/kg administered intraperitoneally), was administered in 10, 60 and 120 min (ET and GT) or 60 and 120 min (MB) after the poisoning with SFA, having the K_{LD50} on the level of 3.3 (Table 2).

TABLE 2
Assessment of the effectiveness of methylene blue (MB),
ethanol (ET) and Glycerol Trinitrate (GT) combination
under acute SFA intoxication of female rats

| | | Number | | Probit analysis |
|---|---|---|---|---|
| Dose, | | of | Death | using Litchfield- |
| mg/kg | Therapy | rats | rate | Wilcoxon method |
| 3.0 | No | 10 | 0 | LD16 = 1.44 |
| 4.0 | No | 16 | 7 | LD50 = 4.33 |
| 5.0 | No | 16 | 9 | (3.32 ÷ 5.24) |
| 6.0 | No | 18 | 16 | LD84 = 7.22 |
| 8.0 | No | 8 | 8 | |
| 10.0 | MB 1 h + 2 h | 6 | 1 | LD16′ = 11.34 |
| | after poisoning | | | LD50′ = 14.23 |
| | ET + GT 10\grave{ } + 1 h + 2 h | | | (12.45 ÷ 16.01) |
| | after poisoning | | | LD84′ = 17.11 |
| 13.0 | MB 1 h + 2 h | 6 | 1 | |
| | after poisoning | | | |
| | ET + GT 10\grave{ } + 1 h + 2 h | | | |
| | after poisoning | | | |
| 16.0 | MB 1 h + 2 h | 6 | 4 | |
| | after poisoning | | | |
| | ET + GT 10\grave{ } + 1 h + 2 h | | | |
| | after poisoning | | | |
| 18.0 | MB 1 h + 2 h | 6 | 6 | |
| | after poisoning | | | |
| | ET + GT 10\grave{ } + 1 h + 2 h | | | |
| | after poisoning | | | |

| Coefficients of efficiency: | K_{LD16} = LD16′/ |
|---|---|
| | LD16 = 7.9 |
| | K_{LD50} = LD50′/ |
| | LD50 = 3.3 |
| | K_{LD84} = LD84′/ |
| | LD84 = 2.4 |

The combination of four compounds: aqueous solutions of methylene blue (MB) (dose 5 mg/kg administered subcutaneously) and 25% ethanol (ET), 0.25% glycerol trinitrate (GT), and 12.5-25 mg/ml sodium glutamate (SG), so that corresponding doses for pure ET, GT, and SG were 1 g/kg, 10 mg/kg, and 50-100 mg/kg, given intraperitoneally (i.p.), was administered in 10, 60 and 120 min, 24 h and 48 h (ET, GT, and SG), 10 and 120 min, 24 h or 48 h (MB) after the poisoning with SFA, having the K_{LD50} at the level of 4.2 (Table 3).

TABLE 3
Assessment of the effectiveness of methylene blue (MB), ethanol
(ET), glycerol trintrate (GT) and sodium glutamate (SG) combination
under acute SFA intoxication of male Wistar rats

| | | Number | | Probit analysis, |
|---|---|---|---|---|
| Dose, | | of | Death | after Litchfield- |
| mg/kg | Therapy | rats | rate | Wilcoxon |
| 1.5 | No | 4 | 1 | LD16 = 1.26 |
| 2.0 | No | 4 | 2 | LD50 = 1.87 |
| 2.5 | No | 4 | 4 | (1.37 ÷ 2.57) |
| 3.0 | No | 4 | 3 | LD84 = 2.78 |
| 7.5 | MB 10\grave{ } + 2 h + 24 h + | 7 | 3 | LD16′ = 6.42 |
| | 48 h after poisoning; | | | LD50′ = 7.87 |
| | ET + GT + SG | | | (6.99 ÷ 8.86) |
| | 10\grave{ } + 1 h + 2 h + 24 h + | | | LD84′ = 9.66 |
| | 48 h after poisoning | | | |
| 8.0 | MB 10\grave{ } + 2 h + 24 h + | 8 | 5 | |
| | 48 h after poisoning; | | | |
| | ET + GT + SG | | | |
| | 10\grave{ } + 1 h + 2 h + 24 h + | | | |
| | 48 h after poisoning | | | |
| 9.0 | MB | 8 | 4 | |
| | 10\grave{ } + 2 h + 24 h + | | | |
| | 48 h after poisoning; | | | |
| | ET + GT + SG | | | |
| | 10\grave{ } + 1 h + 2 h + 24 h + | | | |
| | 48 h after poisoning | | | |
| 10.0 | MB | 4 | 4 | |
| | 10\grave{ } + 2 h + 24 h + | | | |
| | 48 h after poisoning; | | | |
| | ET + GT + SG | | | |
| | 10\grave{ } + 1 h + 2 h + 24 h + | | | |
| | 48 h after poisoning | | | |

| Coefficients of efficiency: | K_{LD16} = LD16′/ |
|---|---|
| | LD16 = 5.1 |
| | K_{LD50} = LD50′/ |
| | LD50 = 4.2 |
| | K_{LD84} = LD84′/ |
| | LD84 = 3.5 |

To test the effectiveness of the different preparations under acute intoxication with ethylene glycol (EG) and potassium cyanide (KCN), the combination of methylene blue (MB), ethanol (ET) and glycerol trinitrate (GT) alone and in addition to 4-methylpyrazole (4 MP) was used. Pilot experiments were conducted to find out which of the two combinations (MB+SG or MB+ET+GT) was more effective as a universal remedy. Data (not shown) demonstrated that the combination of MB+ET+GT was more effective. Subsequent experiments were conducted to collect statistically relevant data to calculate the coefficient of efficiency (K) and the coefficient of guaranteed defense (KG) with MB+ET+GT.

The combinations of MB+ET+GT alone, 4-methylpyrazole alone and the combined application of MB+ET+GT and 4-methylpyrazole were tested following poisoning with ethylene glycol, administered subcutaneously. 4-methylpyrazole was also administered subcutaneously (but at a different place of the body) at a dose of 10 mg/kg, 10 mins and 2 hours after poisoning. To test the combination MB+ET+GT without 4-methylpyrazole, the combination of aqueous solution of MB (dose 5 mg/kg administered subcutaneously) and aqueous solution of 50% ET and 0.5% GT (corresponding doses for pure ET and GT were 1 g/kg and 10 mg/kg administered intraperitoneally), was administered in 10 and 120 min (for MB) and 10, 60 and 120 min (for ET and GT) after poisoning with ethylene glycol (EG). To test the combination MB+ET+GT with 4-methylpyrazole, the combination of aqueous solution of MB (dose 5 mg/kg administered subcutaneously) and aqueous solution of 50% ET and 0.5% GT (corresponding doses for pure ET and GT were 1 g/kg and 10 mg/kg administered intraperitoneally), was administered in 1 h and 24 h (for MB, ET and GT) after poisoning with ethylene glycol (EG). The results of the experiments with ethylene glycol are shown in Table 4.

The combination of MB+ET+GT had a good coefficient of efficiency at the level of 2.29, but 4 MP was more specific with K=3.71 and KG=2.04. A combined administration of MB+ET+GT and 4 MP increased the K up to 4.0 and the KG up to 2.19, which demonstrated the effectiveness administration of MB+ET+GT alone or in combination with the recognized antidote if the latter is available.

In case of potassium cyanide (KCN) poisoning, the effectiveness of the combination of methylene blue (MB), ethanol (ET) and glycerol trinitrate (GT) was tested with slight modifications. Methylene blue (MB) and ethanol with glycerol trinitrate (GT) were mixed together and introduced intraperitoneally 1 and 30 min after the poisoning and the dose of methylene blue (MB) was increased to 10 mg/kg. The experimental results obtained with KCN can be seen in Table 5.

To prove the effectiveness of the various combinations in ischemic states, acute ammonia intoxication was used as a model of acute encephalopathy (hepatic coma). Experiments were conducted with Wistar rats and outbred mice. Solutions of NH₄Cl (pH 7.4) and other compounds were introduced intraperitoneally, but not in the same place—on the left and on the right sides. Because the lethal dose of NH₄Cl decreases if applied in concentrated solutions, in all the following experiments, the volume of 0.4 M NH₄Cl introduced was 0.5-0.7 ml per mouse. The death volume (LD100) of 0.4 M NH₄Cl was found to be 0.5 ml in a 20 g mouse and 5 ml in a 200 g rat which is equivalent to 14 mmol/kg for a mouse and 12 mmol/kg for a rat.

When injecting NH₄Cl at dose 9-10 mmol/kg, the state of coma developed in 8-10 min. 50-60% of animals survived and their motion activity was completely restored in 1-1.5 hours. As a control, the salt solution of the same volume was injected. LD100 was estimated in each experiment. 20 mice were used for evaluation of LD100, the control group (intoxication without therapy) consisted of 30 animals, and the experimental group (intoxication with therapy) also consisted of 30 animals. It should be pointed out that in this experiment, the animals were treated with NH₄Cl at the level of LD110. A combination of methylene blue and sodium glutamate was injected 1 min after the poisoning. The animals’ survival was at the level of 50%.

Further experiments were performed to compare the effectiveness of MB+ET+GT+SG combination with MB+ET+GT+2-oxoglutarate and with MB+ET+GT+pyruvate in male Wistar rats suffering from acute sodium fluoroacetate (SFA) intoxication (See Table 6). The combination of four compounds: aqueous solution of methylene blue (MB) (dose 5 mg/kg administered subcutaneously), and aqueous solution of ethanol (ET), glycerol trinitrate (GT), and sodium glutamate (SG) or sodium pyruvate (SP) or sodium 2-oxoglutarate (SO) in place of glutamate (corresponding doses of ET was 1 g/kg, GT—10 mg/kg, and SG or SP or SO—75 mg/kg, given intraperitoneally); was administered in 10, 60 and 120 min (for ET, GT, and SG or SP or SO), and 10 and 120 min (for MB) after the poisoning with SFA. The experimental data shown on Table 6 indicates that all three combinations were effective in the treatment of acute sodium fluoracetate intoxication in male Wistar rats. The MB+ET+GT+SG combination had a K_{LD50} of 3.7, the MB+ET+GT+SO combination had a K_{LD50} of 4.0 and the MB+ET+GT+SP combination had a K_{LD50} of 4.3.

TABLE 4
Experimental development of the antidotal therapy under acute intoxication of male
Wistar rats with ethylene glycol (EG)

| | Toxicity parameters of EG, mg/kg | | | | | | | |
|---|---|---|---|---|---|---|---|---|
| Number of rats | LD₁₀ | LD₁₆ | LD₅₀ | LD₈₄ | LD₉₀ | Therapy* | K** | KG*** |
| 65 | 2220 | 2490 | 3500 | 4530 | 5200 | — | — | — |
| | | | (3153 ÷ 3885) | | | | | |
| 24 | 4500 | 5100 | 8000 | 12000 | 13900 | MB | 2.29 | 0.87 |
| | | | (6150 ÷ 10400) | | | 10 + 120 min | | |
| | | | | | | after poisoning | | |
| | | | | | | ET + GT | | |
| | | | | | | 10 + 60 + 120 min | | |
| | | | | | | after poisoning | | |
| 26 | 10600 | 11000 | 13000 | 15200 | 16100 | 4MP | 3.71 | 2.04 |
| | | | (11700 ÷ 14400) | | | 10 + 120 min | | |
| | | | | | | after poisoning | | |
| 20 | 11400 | 12000 | 14000 | 16000 | 17000 | 4MP | 4.00 | 2.19 |
| | | | (12610 ÷ 15540) | | | 10 + 120 min | | |
| | | | | | | after poisoning | | |
| | | | | | | MB + ET + GT | | |
| | | | | | | 1 + 24 h | | |
| | | | | | | after poisoning | | |

*The therapeutic treatment was carried out with MB + ET + GT and/or 4MP.
**K - Coefficient of efficiency (the ratio of LD50 for treated animals to LD50 for non-treated animals);
***KG - Coefficient of guaranteed defense (the ratio of LD10 for treated animals to LD90 for non-treated animals).

TABLE 5
Experimental development of the antidotal therapy under acute
intoxication of male Wistar rats with KCN

| | Toxicity parameters of KCN, mg/kg | | | | | | | |
|---|---|---|---|---|---|---|---|---|
| Number of rats | LD₁₀ | LD₁₆ | LD₅₀ | LD₈₄ | LD₉₀ | Therapy* | K** | KG*** |
| 24 | 7.3 | 7.5 | 8.0 (7.6 ÷ 8.5) | 8.7 | 8.9 | — | — | — |
| 29 | 6.6 | 7.2 | 10.2 (8.5 ÷ 12.2) | 13.9 | 15.6 | MB + ET + GT | 1.28 | 0.74 |
| | | | | | | 1 + 30 min | | |
| | | | | | | after poisoning | | |

*The therapeutic treatment was carried out with MB + ET + GT (a modified version, see text).
**K - Coefficient of Defense (the ratio of LD50 for treated animals to LD50 for non-treated animals);
***KG - Coefficient of Guaranteed Defense (the ratio of LD10 for treated animals to LD90 for non-treated animals).

TABLE 6
A comparative study of methylene blue (MB), ethanol
(ET), glycerol trintrate (GT) with sodium glutamate
(SG) or 2-oxoglutarate or pyruvate combination under
acute SFA intoxication of male Wistar rats

| | | Number | | Probit analysis, |
|---|---|---|---|---|
| Dose, | | of | Death | after Litchfield- |
| mg/kg | Therapy | rats | rate | Wilcoxon |
| 1.5 | No | 1 | 0 | LD16 = 1.44 |
| 1.8 | No | 4 | 1 | LD50 = 2.03 |
| 2.0 | No | 13 | 6 | (1.70 ÷ 2.42) |
| 2.25 | No | 6 | 4 | LD84 = 2.84 |
| 2.5 | No | 4 | 3 | |
| 7.0 | MB 10\grave{ } + 2 h | 4 | 1 | LD16′ = 5.86 |
| | after poisoning; | | | LD50′ = 7.57 |
| | ET + GT + SG | | | (6.79 ÷ 8.43) |
| | 10\grave{ } + 1 h + 2 h | | | LD84′ = 9.76 |
| | after poisoning | | | |
| 8.0 | MB 10\grave{ } + 2 h | 4 | 3 | |
| | after poisoning; | | | |
| | ET + GT + SG | | | |
| | 10\grave{ } + 1 h + 2 h | | | |
| | after poisoning | | | |
| 9.0 | MB 10\grave{ } + 2 h | 18 | 15 | |
| | after poisoning; | | | |
| | ET + GT + SG | | | |
| | 10\grave{ } + 1 h + 2 h | | | |
| | after poisoning | | | |
| 10.0 | MB 10\grave{ } + 2 h | 17 | 13 | |
| | after poisoning; | | | |
| | ET + GT + SG | | | |
| | 10\grave{ } + 1 h + 2 h | | | |
| | after poisoning | | | |

| | |
|---|---|
| Coefficients of efficiency | K_{LD16} = LD16′/ |
| | LD16 = 4.1 |
| | K_{LD50} = LD50′/ |
| | LD50 = 3.7 |
| | K_{LD84} = LD84′/ |
| | LD84 = 3.4 |

MB + ET + GT + sodium 2-oxoglutarate
75 mg/kg, - in place of glutamate
10\grave{ } + 1 h + 2 h after SFA intoxication

| | | | | |
|---|---|---|---|---|
| 8.0 | MB 10\grave{ } + 2 h | 4 | 1 | LD16′ = 7.34 |
| | after poisoning; | | | LD50′ = 8.27 |
| | ET + GT + SO | | | (7.01 ÷ 9.76) |
| | 10\grave{ } + 1 h + 2 h | | | LD84′ = 9.32 |
| | after poisoning | | | |
| 9.0 | MB 10\grave{ } + 2 h | 4 | 4 | |
| | after poisoning; | | | |
| | ET + GT + SO | | | |
| | 10\grave{ } + 1 h + 2 h | | | |
| | after poisoning | | | |
| 10.0 | MB 10\grave{ } + 2 h | 8 | 7 | |
| | after poisoning; | | | |
| | ET + GT + SO | | | |
| | 10\grave{ } + 1 h + 2 h | | | |
| | after poisoning | | | |

| | |
|---|---|
| Coefficients of efficiency | K_{LD16} = LD16′/ |
| | LD16 = 5.1 |
| | K_{LD50} = LD50′/ |
| | LD50 = 4.0 |
| | K_{LD84} = LD84′/ |
| | LD84 = 3.3 |

MB + ET + GT + sodium pyruvate
75 mg/kg, - in place of glutamate
10\grave{ } + 1 h + 2 h after SFA intoxication

| | | | | |
|---|---|---|---|---|
| 9.0 | MB 10\grave{ } + 2 h | 7 | 4 | LD16′ = 7.68 |
| | after poisoning; | | | LD50′ = 8.78 |
| | ET + GT + SP | | | (7.93 ÷ 9.74) |
| | 10\grave{ } + 1 h + 2 h | | | LD84′ = 10.04 |
| | after poisoning | | | |
| 10.0 | MB 10\grave{ } + 2 h | 6 | 5 | |
| | after poisoning; | | | |
| | ET + GT + SP | | | |
| | 10\grave{ } + 1 h + 2 h | | | |
| | after poisoning | | | |

| | |
|---|---|
| Coefficients of efficiency | K_{LD16} = LD16′/ |
| | LD16 = 5.1 |
| | K_{LD50} = LD50′/ |
| | LD50 = 4.3 |
| | K_{LD84} = LD84′/ |
| | LD84 = 3.5 |

A number of embodiments of the invention have been described. Nevertheless, it will be understood that various modifications may be made without departing from the spirit and scope of the invention. Accordingly, other embodiments are within the scope of the following claims:

1. A pharmaceutical composition comprising a phenothiazine compound, a dioic acid compound and a pharmaceutically acceptable carrier.

2. The pharmaceutical composition of claim 1, wherein the phenothiazine compound is methylene blue.

3. The pharmaceutical composition of claim 1, wherein the dioic acid compound is a glutamate salt.

4. The pharmaceutical composition of claim 3, wherein the glutamate salt is sodium glutamate.

5. A pharmaceutical composition comprising a phenothiazine compound, a nitroester compound, ethanol and a pharmaceutically acceptable carrier.

6. The pharmaceutical composition of claim 5, wherein the phenothiazine compound is methylene blue.

7. The pharmaceutical composition of claim 5, wherein the nitroester compound is glycerol trinitrate.

8. The pharmaceutical composition of claim 5, further comprising a pyrazole.

9. The pharmaceutical composition of claim 8, wherein the pyrazole is 4-methylpyrazole.

10. The pharmaceutical composition of claim 5, further comprising a dioic acid compound.

11. The pharmaceutical composition of claim 10, wherein the dioic acid compound is a glutamate salt, an aspartate salt, or a succinate salt.

12. The pharmaceutical composition of claim 11, wherein the dioic acid compound is sodium glutamate or a 2-oxoglutrate salt.

13. (canceled)

14. The pharmaceutical composition of claim 12, wherein the 2-oxoglutrate salt is sodium 2-oxoglutrate.

15. (canceled)

16. (canceled)

17. The pharmaceutical composition of claim 5, further comprising a monocarboxylic acid compound.

18. The pharmaceutical composition of claim 17, wherein the monocarboxylic acid compound is a pyruvate salt.

19. The pharmaceutical composition of claim 18, wherein the pyruvate salt is sodium pyruvate.

20. A method of treating fluoroacetate intoxication, ethylene glycol intoxication or cyanate intoxication comprising administering to a subject in need thereof an effective amount of a composition comprising a phenothiazine compound, a nitroester compound and ethanol.

21.-32. (canceled)

33. The method of claim 20, wherein the phenothiazine compound is administered subcutaneously.

34. The method of claim 20, wherein the phenothiazine compound is administered 45-75 minutes and 100-140 minutes after intoxication.

35. The method of claim 20, wherein the phenothiazine compound is administered 5-15 minutes and 100-140 minutes after intoxication.

36. The method of claim 20, wherein the nitroester compound and ethanol are administered intraperitoneally.

37. The method of claim 20, wherein the nitroester compound and ethanol are administered 5-15 minutes, 50-70 minutes and 100-140 minutes after intoxication.

38. The method according to claim 20, wherein the phenothiazine compound, the nitroester compound and ethanol are further administered 20-28 hours and 40-56 hours after intoxication.

39.-47. (canceled)

48. The method of claim 20, wherein the composition further comprises a pyrazole.

49. The method of claim 48, wherein the pyrazole is 4-methylpyrazole.

50. The method of claim 48, wherein the pyrazole is administered subcutaneously.

51. The method of claim 48, wherein the pyrazole is administered 5-15 minutes and 100-140 minutes after intoxication.

52.-63. (canceled)
